(19) United States
(12) Patent Application Publication
(10) Pub. No.: US 2014/0066764 A1
(43) Pub. Date: Mar. 6, 2014

(54) CHARACTERIZATION OF TISSUE BY ULTRASOUND ECHOGRAPHY

(71) Applicant: Boston Scientific Scimed Inc., Maple Grove, MN (US)

(72) Inventors: Raj Subramaniam, Fremont, CA (US); Steven T. Onishi, Cupertino, CA (US)

(73) Assignee: Boston Scientific Scimed Inc., Maple Grove, MN (US)

(21) Appl. No.: 14/018,386

(22) Filed: Sep. 4, 2013

Related U.S. Application Data
(60) Provisional application No. 61/697,122, filed on Sep. 5, 2012.

(51) Int. Cl. A61B 8/12

(52) U.S. Cl. CPC A61B 8/12
USPC 600/438

(57) ABSTRACT

Various embodiments concern sensing a first signal indicative of a plurality of different phases of a cardiac cycle with a sensor and sensing a second signal with an ultrasound sensor within the heart over different phases, the second signal indicative of the density of a section of cardiac tissue. Each phase can be associated with an indication of the density of the section of cardiac tissue during the phase based on the second signal. It can be determined whether the section of cardiac tissue compressed during the cardiac cycle based on a change in the indication of density of the cardiac tissue over the plurality of different phases. The efficacy of ablation therapy can be evaluated based on the compressibility of the section of cardiac tissue.

### CROSS-REFERENCE TO RELATED APPLICATION

This application claims priority to U.S. Provisional Application No. 61/697,122, filed Sep. 5, 2012, which is herein incorporated by reference in its entirety.

### TECHNICAL FIELD

The present disclosure relates generally to analyzing anatomical structures within the body. More specifically, the present disclosure relates to devices, systems, and methods for characterizing tissue properties using gated ultrasonic echography.

### BACKGROUND

In ablation therapy, it is often necessary to determine various characteristics of body tissue at a target ablation site within the body. In interventional cardiac electrophysiology (EP) procedures, for example, it is often necessary for the physician to determine the condition of cardiac tissue at a target ablation site in or near the heart. During some EP procedures, the physician may deliver a mapping catheter through a main vein or artery into an interior region of the heart to be treated. Using the mapping catheter, the physician may then determine the source of a cardiac rhythm disturbance or abnormality by placing a number of mapping elements carried by the catheter into contact with the adjacent cardiac tissue and then operating the catheter to generate an electrophysiology map of the interior region of the heart based on sensed electrical cardiac signals. Once a map of the heart is generated, the physician may then advance an ablation catheter into the heart, and position an ablation electrode carried by the catheter tip near the targeted cardiac tissue to ablate the tissue and form a lesion, thereby treating the cardiac rhythm disturbance or abnormality. In some techniques, the ablation catheter itself may include a number of mapping electrodes, allowing the same device to be used for both mapping and ablation.

Various ultrasound-based imaging catheters and probes have been developed for visualizing body tissue in applications such as interventional cardiology, interventional radiology, and electrophysiology. For interventional cardiac electrophysiology procedures, for example, ultrasound imaging devices have been developed that permit the visualization of anatomical structures of the heart directly and in real-time. In some electrophysiology procedures, for example, ultrasound catheters may be used to image the intra-atrial septum, to guide transseptal crossing of the atrial septum, to locate and image the pulmonary veins, and to monitor the atrial chambers of the heart for signs of a perforation and pericardial effusion.

### SUMMARY

The present disclosure relates to devices, systems, and methods for imaging and characterizing tissue properties using gated ultrasonic echography.

In example 1, a system comprises at least one catheter having a distal end configured to be introduced into the heart; at least one ultrasound sensor on the distal end of the at least one catheter, the at least one ultrasound sensor configured to output a first signal indicative of the intensity of ultrasound energy received by the ultrasound sensor from a section of cardiac tissue, the intensity of the ultrasound energy indicative of the density of the section of cardiac tissue; a sensor configured to output a second signal indicative of a plurality of different phases of at least one cardiac cycle; and control circuitry configured to associate each phase of the plurality of different phases of the at least one cardiac cycle with the intensity level of ultrasound energy received by the ultrasound sensor from the section of cardiac tissue during the phase, determine whether the section of cardiac tissue compressed during the at least one cardiac cycle based on a difference between the intensity levels of ultrasound energy associated with different phases of the at least one cardiac cycle, and generate an output based on the determination of whether the section of cardiac tissue compressed.

In example 2, the system of example 1, wherein the control circuitry is configured to determine that the section of cardiac tissue compressed if the intensity level of ultrasound energy associated with a systolic phase is greater relative to the intensity level of ultrasound energy associated with a diastolic phase and that the section of cardiac tissue did not compress if the intensity level of ultrasound energy associated with the systolic phase is similar to the intensity level of ultrasound energy associated with the diastolic phase.

In example 3, the system of either example 1 or 2, wherein the difference between the intensity levels of ultrasound energy associated with different phases of the at least one cardiac cycle is indicative of a change in density of the section of cardiac tissue between the different phases of the at least one cardiac cycle.

In example 4, the system of any of examples 1-3, further comprising a display, wherein the control circuitry is configured to generate an indication on the display of the state of the section of cardiac tissue based on the determination of whether the section of cardiac tissue compressed.

In example 5, the system of any of examples 1-3, wherein the control circuitry is configured to generate a cardiac map on the display and highlight the section of cardiac tissue on the cardiac map based on the control circuitry determining that the section did not compress.

In example 6, the system of any of examples 1-5, further comprising an ablation element configured to output a cardiac ablation therapy.

In example 7, the system example 6, wherein the control circuitry is configured to determine whether the section of cardiac tissue was ablated by the cardiac ablation therapy based on the determination of whether the section of cardiac tissue compressed.

In example 8, the system of either of examples 6 or 7, wherein the control circuitry is configured to repeatedly or continuously deliver the cardiac ablation therapy with the ablation element to the section of cardiac tissue until the control circuitry determines that the section of cardiac tissue no longer compresses.

In example 9, the system of any of examples 1-8, wherein: the first signal is indicative of the level of ultrasound energy received by the ultrasound sensor from an additional section of cardiac tissue that is adjacent to the first section; and the control circuitry is configured to associate each phase of the plurality of different phases of the at least one cardiac cycle with the intensity level of ultrasound energy received by the at least one ultrasound sensor from the additional section of cardiac tissue during the phase and determine whether the section of cardiac tissue compressed during the at least one cardiac cycle relative to the additional section of cardiac tissue based on the intensity levels of ultrasound energy associated with the different phases of each of the section and the additional section of cardiac tissue.

In example 10, the system of any of examples 1-9, wherein the control circuitry is configured to process the first signal in accordance with A-mode ultrasound operation.

In example 11, the system of any of examples 1-10, wherein the control circuitry is configured to selectively sample the first signal only during respective portions of the plurality of different phases based on the second signal to associate each phase of the plurality of different phases with the intensity level of ultrasound energy received by the ultrasound sensor during the respective portion of the phase.

In example 12, the system of any of examples 1-11, wherein the control circuitry is configured to reduce or eliminate changes in the intensity level of the ultrasound energy of the first signal due to wall motion of the section of cardiac tissue.

In example 13, a method of assessing cardiac ablation, the method comprising sensing a first signal indicative of a plurality of different phases of at least one cardiac cycle with a sensor; sensing a second signal with an ultrasound sensor within the heart over the plurality of different phases of the at least one cardiac cycle, the second signal indicative of the density of a section of cardiac tissue; associating each phase of the plurality of different phases of the at least one cardiac cycle with an indication of the density of the section of cardiac tissue during the phase based on the second signal; determining whether the section of cardiac tissue compressed during the at least one cardiac cycle based on a change in the indication of density of the section of cardiac tissue over the plurality of different phases of the at least one cardiac cycle.

In example 14, the method of example 13, further comprising delivering an ablation therapy to a section of cardiac tissue, the ablation therapy delivered by a catheter to the section of cardiac tissue and determining whether the section of cardiac tissue was ablated by the delivery of the ablation therapy based on whether the section of cardiac tissue compressed during the at least one cardiac cycle.

In example 15, the method of either of examples 13 or 14, wherein determining whether the section of cardiac tissue compressed comprises: determining that the section compressed if the density of the section associated with the systolic phase is indicated to be greater than the density of the section associated with the diastolic phase; and determining that the section did not compress if the density of the section associated with the systolic phase is indicated to be similar to the density of the section associated with the diastolic phase.

In example 16, the method of either of examples 14 or 15, further comprising redelivering the ablation therapy to the section of cardiac tissue if it is determined that the section of cardiac tissue was not ablated by the delivery of the ablation therapy.

In example 17, the method of any of examples 13-16, wherein the plurality of different phases of the at least one cardiac cycle comprises at least a diastole phase and a systole phase.

In example 18, the method of any of examples 13-17, wherein sensing the second signal comprises selectively sensing the second signal only during respective portions of the plurality of different phases based on the first signal to associate each phase of the plurality of different phases with the indication of the density of the section of tissue during the phase.

In example 19, the method of any of examples 13-18, wherein the density of the section of cardiac tissue is indicated by the intensity level of ultrasound energy received by the second sensor as reflected from the section of cardiac tissue.

In example 20, a system comprising at least one catheter having a distal end configured to be introduced into the heart; at least one ultrasound sensor on the distal end of the at least one catheter, the at least one ultrasound sensor configured to output a first signal indicative of the intensity level of ultrasound energy received by the ultrasound sensor from a section of cardiac tissue, the intensity level of ultrasound energy received by the at least one ultrasound sensor indicative of the density of the section of cardiac tissue; a sensor configured to output a second signal indicative of a plurality of different phases of at least one cardiac cycle; a display; and control circuitry configured to associate each phase of the plurality of different phases of the at least one cardiac cycle with the intensity level of ultrasound energy received by the at least one ultrasound sensor from the section of cardiac tissue within the phase and generate an output on the display representing the intensity levels of ultrasound energy as associated with the different phases of the plurality of different phases of the at least one cardiac cycle, the output on the display indicative of whether the section of cardiac tissue compressed during the at least one cardiac cycle.

In example 21, the system of example 20, wherein the output on the display comprises overlaid signal traces, each signal trace of the overlaid signal traces representing the intensity level of ultrasound energy as associated with a respective phase of the plurality of different phases of the at least one cardiac cycle.

In example 22, the system of either of examples 21 or 22, wherein the control circuitry is further configured to label at least one phase of the plurality of different phases as a systolic phase and label at least one other phase of the plurality of different phases as a diastolic phase in the output generated on the display based on the second signal.

While multiple embodiments are disclosed, still other embodiments of the present invention will become apparent to those skilled in the art from the following detailed description, which shows and describes various illustrative embodiments of the present disclosure. Accordingly, the drawings and detailed description are to be regarded as illustrative in nature and not restrictive.

### BRIEF DESCRIPTION OF THE DRAWINGS

FIG. 1 shows an exemplary system for characterizing cardiac tissue in accordance with various aspects of this disclosure;

FIG. 2 shows a block diagram of components for characterizing cardiac tissue in accordance with various aspects of this disclosure;

FIG. 3 shows a series of plots characterizing cardiac tissue in accordance with various aspects of this disclosure;

FIG. 4 shows an overlay of ultrasound information for characterizing cardiac tissue in accordance with various aspects of this disclosure;

FIG. 5 shows a flowchart of a method for characterizing cardiac tissue and controlling an ablation therapy in accordance with various aspects of this disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

Various cardiac abnormalities can be attributed to improper electrical activity of cardiac tissue. Such improper electrical activity can include, but is not limited to, generation of electrical signals, conduction of electrical signals, and/or compression of the tissue in a manner that does not support efficient and/or effective cardiac function. For example, an area of cardiac tissue may become electrically active prematurely or otherwise out of synchrony during the cardiac cycle, thereby causing the cardiac cells of the area and/or adjacent areas to contract out of rhythm. The result is an abnormal cardiac contraction that is not timed for optimal cardiac output. In some cases, an area of cardiac tissue may provide a faulty electrical pathway (e.g., a short circuit) that causes an arrhythmia, such as atrial fibrillation or supraventricular tachycardia. In some cases, inactivate tissue (e.g., scar tissue) may be preferable to malfunctioning cardiac tissue.

Cardiac ablation is a procedure by which cardiac tissue is treated to inactivate the tissue. The tissue targeted for ablation may be associated with improper electrical activity, as described above. Cardiac ablation can lesion the tissue and prevent the tissue from improperly generating or conducting electrical signals. For example, a line, a circle, or other formation of lesioned cardiac tissue can block the propagation of errant electrical signals. In some cases, cardiac ablation is intended to cause the death of cardiac tissue and to have scar tissue reform over the lesion, where the scar tissue is not associated with the improper electrical activity. Lesioning therapies include electrical ablation, radiofrequency ablation, cyroablation, microwave ablation, laser ablation, and surgical ablation, among others.

In some cases, it can be difficult to assess the functionality of an area of tissue after the tissue is treated. Although ultrasonic catheters are used for acquiring high resolution images of anatomical structures within the body, such devices often do not provide information associated with the characteristics of the tissue being imaged. For example, normal cardiac tissue, ablated cardiac tissue, and cardiac tissue with edema each have similar ultrasound echogenicity characteristics, and tend to be isoechogenic in their relaxed or uncompressed states. As a result of this attribute, it is often difficult to monitor lesion formation during an ablation procedure, to confirm the transmurality or depth of the lesion, to identify infracted or fibrous tissue, to look for edema near an ablation site, and/or to gather other information associated with the characteristics of the tissue being analyzed.

Characterizing the electrophysiology properties of tissue can also be inadequate in some cases. For example, an electrophysiology study can identify tissue associated with improper electrical activity and, following lesioning, can determine whether the tissue continues to be associated with improper electrical activity. Conventionally, an ablation treatment may be considered successful if the electrophysiology catheter no longer senses improper electrical activity from a particular section of tissue following lesioning. However, the lesioned tissue may merely be stunned or temporarily non-conductive. It may be difficult to distinguish between fully ablated tissue with no conduction and tissue that is rendered nonconductive due to edema. In these cases, the cessation of improper electrical activity may only be temporary, and the improper electrical activity may return later. Edema, for example, can temporarily block improper electrical activity following lesioning, where the improper electrical activity resumes once the edema subsides. In some cases, a surface layer of fibrous tissue may insulate an underlying conductive layer of tissue for a particular area. Excessive treatment of the tissue, on the other hand, may risk the ablation of more tissue then intended and consequently inactivating more tissue then intended, possibly degrading output capabilities.

The present disclosure concerns, among other things, methods, devices, and systems for determining the compressibility of tissue to assess the functionality of the tissue. For example, various embodiments concern determining whether a section of cardiac tissue compresses during a cardiac cycle based on changes in the density of the section of tissue over the cardiac cycle. While normal cardiac tissue, ablated tissue, and tissue with edema all have the same or very similar ultrasound echogenicity, the compressibility of these tissue states are different. The present disclosure discusses exploiting the variability in the compressibility to differentiate these and other tissue states. Changes in density of the tissue during a cardiac cycle can be indicative of tissue that is contracting as part of a cardiac cycle, while fully ablated tissue will not change in density during the cardiac cycle. The change in density of the tissue over the cardiac cycle can be detected by a change in the intensity of an ultrasound echo reflected from the cardiac tissue. Information regarding whether the section of cardiac tissue compresses during a cardiac cycle can be used to determine whether the tissue is healthy (e.g., compressing in rhythm with surrounding tissue), whether the tissue should be lesioned (e.g., for the first time or an additional time), and/or whether the tissue was successfully ablated in a previous treatment, among other things. Various further embodiments concern guiding ablation therapy based on the compressibility of tissue over the cardiac cycle.

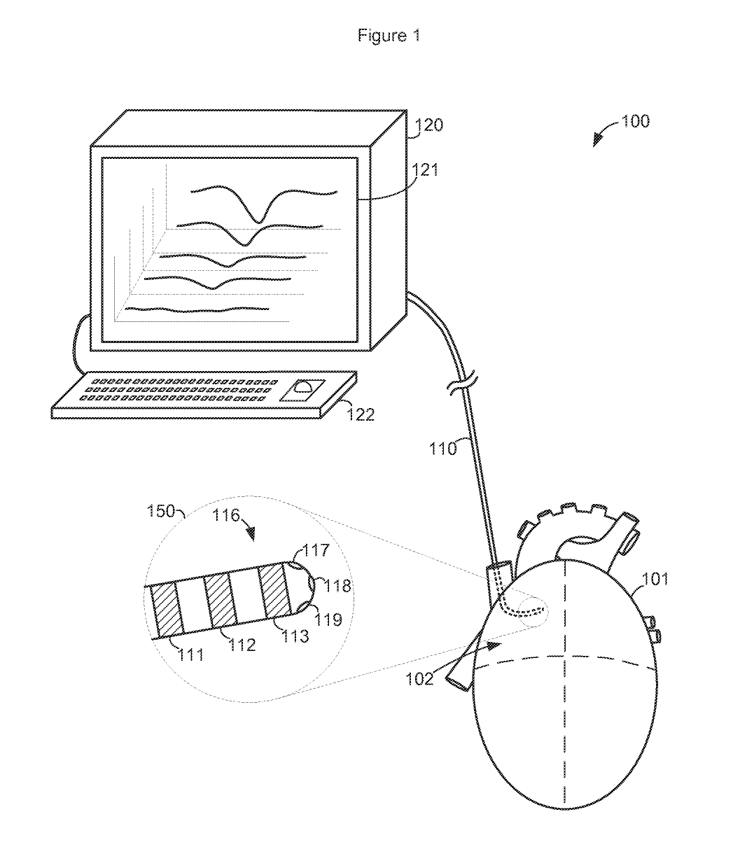

FIG. 1 is an illustrative embodiment of a system 100 for characterizing and ablating cardiac tissue. The system 100 includes a catheter 110 connected to a control unit 120. The catheter 110 can comprise an elongated tubular member having a distal end 116 configured to be introduced within a heart 101 or other area of the body. As shown in FIG. 1, the distal end 116 of the catheter 110 is within the right atrium 102.

As shown in the window 150 of FIG. 1, the distal end 116 of the catheter 110 includes electrodes 111-113. The electrodes 111-113 can be configured for sensing signals, such as electrical cardiac signals. The electrodes 111-113 can additionally or alternatively be used to deliver ablative energy to cardiac tissue. Although three electrodes are illustrated in FIG. 1, various embodiments can have a lesser or greater number of electrodes. Furthermore, electrodes in various other embodiments can be multi-functional (e.g., sensing cardiac signals and delivering ablation therapy) or can have dedicated functionality (e.g., sensing or ablation only).

The distal end 116 of the catheter 110 can further include ultrasound transducers 117-119. The ultrasound transducers can be used for characterizing cardiac tissue, as will be discussed further herein. Ultrasound transducers 117-119 can send ultrasound waves in a pulsing mode and receive ultrasound waves reflected from tissue in a sensing mode. When excited electrically in a pulsing mode, the ultrasound transducers can create pressure waves which travel into the surrounding environment. In the sensing mode, the ultrasound transducers can produce an electrical signal as a result of receiving acoustic waves reflected back to the ultrasound transducers from tissue, which can be processed and displayed on the display 121 of the control unit 120. In various embodiments, an ultrasound sensor is configured to deliver acoustic waves at a frequency greater than about 20 MHz (e.g., in a near field application) from the distal tip of the catheter 110. Ultrasound transducers can be mounted on the exterior of the catheter 110 or may be housed within the body of the catheter 110, where the ultrasound waves are sent and received through the housing of the catheter 110. Each ultrasound transducer can have multi-functionality (e.g., sending and sensing ultrasound energy) in some embodiments, while each ultrasound transducer in some other embodiments may have dedicated functionality (e.g., transmitting or sensing ultrasound energy). In various embodiments, the ultrasound transducers comprise piezoelectric elements formed of a polymer such as PVDF or a piezoceramic material such as PZT. Although three ultrasound transducers are illustrated in FIG. 1, various embodiments can have a lesser or greater number of ultrasound transducers.

The catheter 110 can include one or more lumens having conductors and/or other elements facilitating the transmission of signals, fluids, etc. along the catheter 110. Other members can also be moved through the catheter 110 within the one or more lumens, such as a guidewire or tendon for distal end 116 articulation. A knob on a handle (not illustrated) of the catheter 110 may be used to articulate the distal end 116 of the catheter 110 so that the distal end 116 and the electrodes 111-113 can be moved along various sections of cardiac tissue. The catheter 100 can be connected to one or more extensions proximally for bridging to the control unit 120.

In various embodiments, ultrasound transducers are arranged in a phased array on the distal end 116 of the catheter 110. For example, a plurality of ultrasound transducers can be arranged in a line or other formation and can be sequentially activated. In some embodiments, a single rotating or otherwise moving ultrasound transducer may be provided inside the catheter 110 to scan an area of tissue, although multiple rotating or otherwise moving ultrasound transducers can also be provided. The system 100 is capable of acquiring and processing ultrasound signals in multiple modes simultaneously or sequentially. Ultrasound modes can include M-mode, A-Mode, and/or B-mode, which are further described herein.

The control unit 120 of the system 100 includes a display 121 (e.g., LCD) for displaying information. The control unit 120 further includes a user input 122 which can comprise one or more buttons, toggles, a track ball, a mouse, or the like for providing user input. The control unit 120 can comprise a hardware console and software system for collecting and processing information as discussed herein for characterizing tissue. The control unit 120 can contain control circuitry for performing the functions referenced herein.

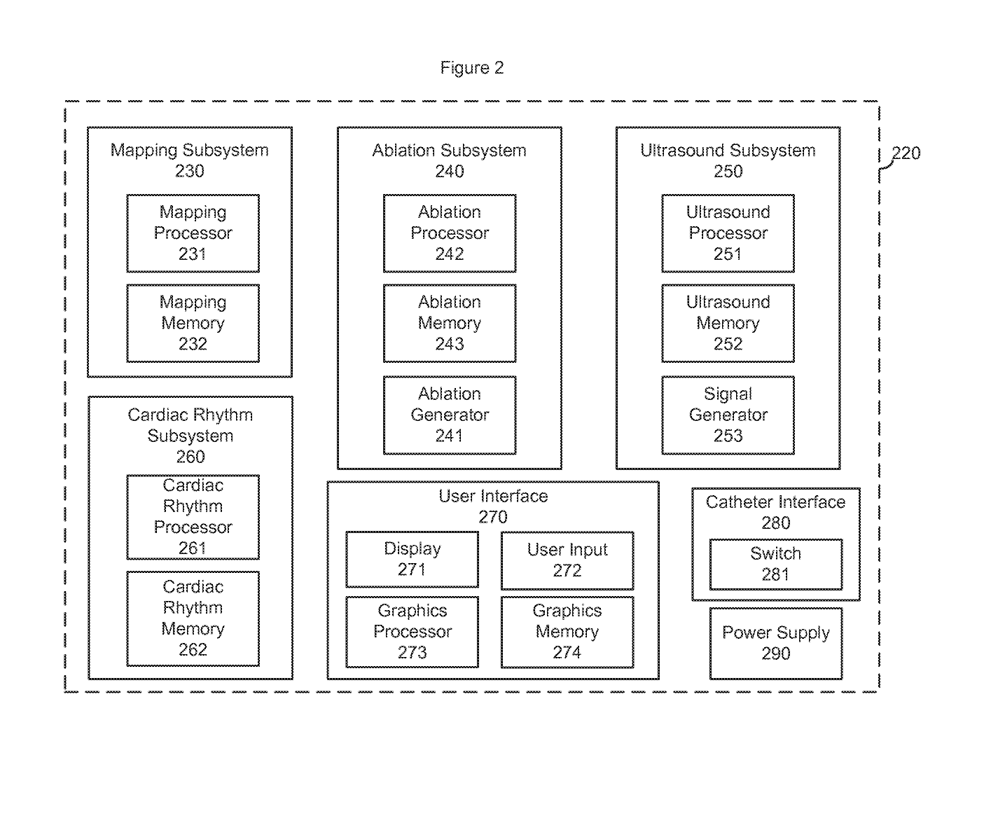

FIG. 2 illustrates a block diagram showing control circuitry and other components for performing functions referenced herein. The control circuitry can be housed within control unit 220, which can comprise a single housing or multiple housings among which components are distributed. The components of the control unit 220 can be powered by a power supply 290 which can supply electrical power to any of the components of the control unit 220 and the system 100. The power supply 290 can plug into an electrical outlet and/or provide power from a battery.

The block diagram of FIG. 2 illustrates a mapping subsystem 230 which includes components for operating the mapping functions of the system. The mapping functions can include sensing one or more cardiac signals from the surface of the heart (e.g., via electrodes 111-113 and one or more conductors within the catheter 110), mapping conduction patterns, identifying unwanted electrical activity, and identifying one or more target sites within the body, among other things. Target sites can include sections of cardiac tissue that support aberrant conductive pathways in the heart or are associated with improper cardiac function. The mapping processor 231 can be configured to execute program instructions stored in the mapping memory 232 to derive activation times and voltage distribution from the electrical signals obtained from the electrodes 111-113 to identify irregular electrical signals within the heart and/or perform other functions. The cardiac information can then be graphically displayed as a map on the display 271. An example mapping system that can be employed to detect electrical signals in myocardial tissue for use in identifying target treatment sites and/or for providing ablation energy to target sites is further described in U.S. Pat. No. 7,720,520, which is expressly incorporated herein by reference in its entirety for all purposes. Further details regarding electrophysiology mapping are provided, for example, in U.S. Pat. Nos. 5,485,849, 5,494,042, 5,833,621, and 6,101,409, each of which is expressly incorporated herein by reference in its entirety for all purposes. In some embodiments, 3D mapping functions can be used to track the three dimensional position of the catheter 110. The electrodes 111-113 can be used to make impedance measurements to determine the 3D position of the catheter 110 in the cardiac space. Magnetic fields can also be created and sensed by a sensor within the catheter 110 to determine the 3D position of the catheter 110 in the cardiac space.

The block diagram of FIG. 2 illustrates an ablation subsystem 240 which includes components for operating the ablation functions of the system. The ablation subsystem 240 includes an ablation generator 241. The ablation generator 241 can provide different therapeutic outputs depending on the particular configuration. For example, in the case of radiofrequency ablation, the ablation generator 241 can generate a high frequency alternating current signal to be output through one or more electrodes (e.g., electrodes 111-113), where ablative heat is generated upon application to tissue. Providing ablation energy to target sites is further described, for example, in U.S. Pat. No. 5,383,874 and U.S. Pat. No. 7,720,520, each of which is expressly incorporated herein by reference in its entireties for all purposes. In some other embodiments, the ablation generator 241 can generate microwave energy to be transmitted by a catheter to ablate targeted tissue or a solution that cools to cryoablate the targeted tissue. The ablation generator 241 may support any other type of ablation therapy. The ablation subsystem 240 may include an ablation processor 242 and ablation memory 243 for controlling ablation functions. For example, the ablation memory 243 can contain program instructions executable by the ablation processor 242 for controlling ablation functions as described herein, such as for managing the delivery of ablation energy.

The block diagram further illustrates an ultrasound subsystem 250 which includes components for operating the ultrasound functions of the system. The ultrasound subsystem 250 can include a signal generator 253 configured to generate a signal for ultrasound transmission. For example, the signal generator 253 may generate a signal (e.g., a 20 MHz signal) for transmission along a conductor of the catheter 110 to one or more of the ultrasound transducers 117-119 which can emit ultrasound waves based on the signal. The ultrasound subsystem 250 can include signal processing circuitry (e.g., a high pass filter) configured to filter and process reflected ultrasound signals as received by an ultrasound transducer in a sense mode and conducted to the ultrasound subsystem 250 through a conductor in the catheter 110. Filtering and processing may include filtering out noise frequencies and amplifying the signal among other functions for highlighting and identifying features of the signals indicative of particular tissue characteristics. The ultrasound subsystem 250 may comprise an ultrasound processor 251. The ultrasound processor 251 may perform signal processing functions, as well as perform other functions. For example, the ultrasound memory 252 can contain program instructions executable by the ultrasound processor 251 for performing the functions described herein, including measuring the intensity of reflected ultrasound energy and determining changes in the ultrasound intensity indicative of tissue compression.

The block diagram of FIG. 2 further illustrates a cardiac rhythm subsystem 260. The cardiac rhythm subsystem 260 can contain circuitry for identifying a cardiac rhythm of a patient. Identifying a cardiac rhythm can include, in various embodiments, identifying particular cardiac phases from a sensed cardiac signal. The one or more cardiac signals may be an electrical cardiac signal sensed from an electrode in contact with the surface of a heart (e.g., electrodes 111-113). A cardiac signal can also be collected from other locations, such as from implanted electrodes or other sensors not in contact with the heart and/or external electrodes or other sensors. In the case of a sensed electrical cardiac signal, cardiac phases may be identified based on a PQRST cardiac pattern, as further explained herein. A cardiac signal may be generated based on sensed sound, such as cardiac sounds collected by an in vivo or external microphone. Sensed cardiac rhythm information may include blood flow sounds and/or heart valve sounds. A signal indicative of a cardiac rhythm may be sensed by an accelerometer that measures cardiac vibrations or other movement associated with the cardiac cycle. Other cardiac rhythm information relating to the cardiac cycle can also be sensed.

The cardiac rhythm memory 262 can contain program instructions executable by the cardiac rhythm processor 261 to perform the functions described herein, such as measuring changes in a cardiac signal, identifying patterns from a cardiac signal (e.g., template matching to known cardiac phases), identifying different phases of a cardiac cycle from a sensed signal, and associating cardiac rhythm information with ultrasound information. The cardiac rhythm subsystem 260 can include signal processing circuitry configured to filter and process sensed cardiac signals.

The block diagram further illustrates a user interface subsystem 270 which can support user input and output functionality. A display 271 (e.g., a liquid crystal display based screen) can be used to display any indication, plot, determination, and/or any other information referenced herein. A graphics processor 273 and graphics memory 274 may be used to support the display 271 functionality, and may be part of the display 271. A user input 272 can be used to allow a user to input information and make selections, among other things. User input 272 can log key and/or other input entries and route the entries to other circuitry.

A catheter interface 280 can provide a port for connecting the catheter 110 to the control circuitry of the control unit 220. A switch 281 can be used to selectively route signals to and from the different components of the control unit 220 along the conductors of the catheter 110.

Although the block diagram of FIG. 2 illustrates multiple processors and memory units, one or more processors can be used to implement the functions described herein. For example, a single processor could perform the functions of multiple subsystems, and as such the subsystems may share control circuitry. Although different subsystems are presented herein, control circuitry may be divided between greater or lesser numbers of subsystems, which may be housed separately or together. In various embodiments, control circuitry is not distributed between subsystems, but rather is provided as a unified computing system. Whether distributed or unified, the components can be electrically connected to coordinate and share resources to carry out functions.

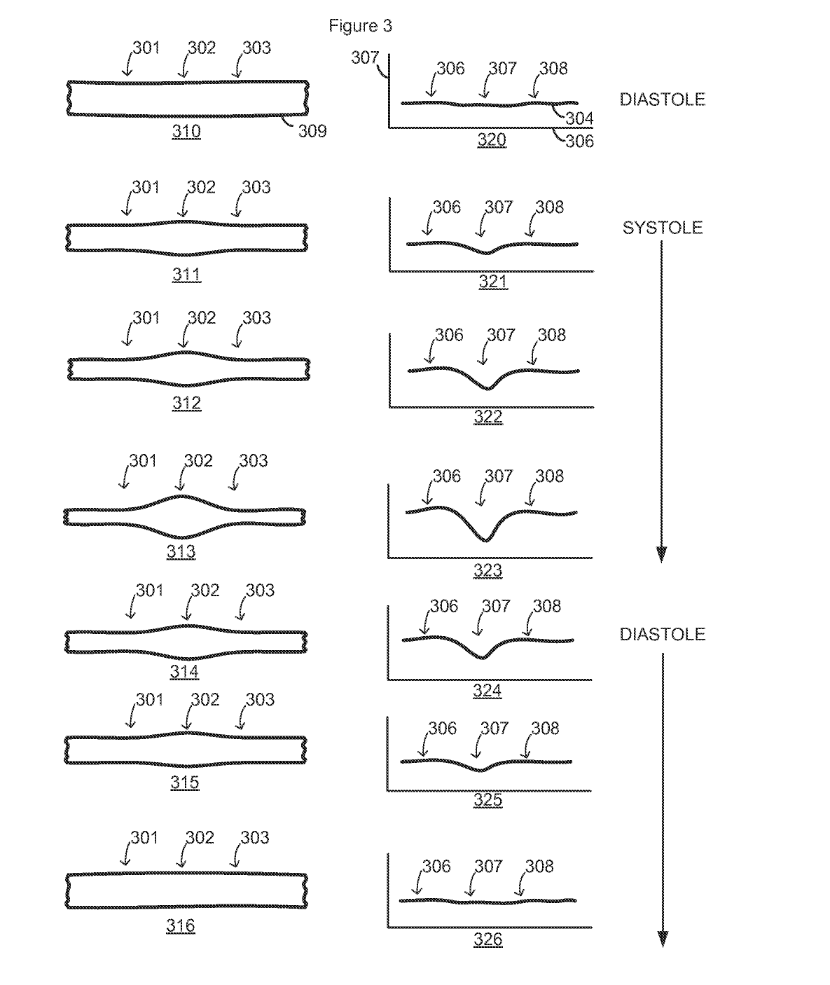

FIG. 3 illustrates a series of contrived plots characterizing cardiac function that can be generated from ultrasound signals. The plots can be generated by control circuitry and displayed on a display in connection with a medical procedure employing ultrasound echography, such as an ablation procedure. Plot 310 shows an area of cardiac tissue 309 having a first section 301, a second section 302, and a third section 303. Plot 310 can be generated by an ultrasound echography system operating according to an M-mode. An ultrasound echography system operating in an M-mode can render moving two dimensional images of tissue in a sectional view. In this case, plot 310 shows a slice through the cardiac tissue 309. The plot 310 can be generated by moving one or more ultrasound sensors along the length of the cardiac tissue (e.g., from the first section 301 to the third section 303), an array of ultrasound sensors that functioning in sequence (e.g., the array spanning from the first section 301 to the third section 303), or by some other technique for collecting near field ultrasound energy from across tissue. Plots 311-316, derived in the same manner as plot 310, show the same section of cardiac tissue 309 at later times in sequence.

Plot 320 represents the same cardiac tissue 309 at essentially the same point in time as shown in plot 310. Plot 320 charts the intensity of the ultrasonic energy signal received by the one or more ultrasound sensors. Plot 320 can be generated by an ultrasound echography system functioning in the A-Mode. An ultrasound echography system operating in A-mode can show the amplitude of the received ultrasound energy. As described herein, the amplitude of the received ultrasound energy can change in proportion with changes in density of the tissue from which the ultrasound waves reflect. An ultrasound echography system operating in M-mode can accordingly show dimensional information while the A-mode function can characterize tissue properties such as density.

The abscissa axis 306 of the plot 320 represents a linear dimension (e.g., over the first, second, and third sections 301-303) across which the ultrasound echography system scans. The ordinate axis 307 represents the intensity of the ultrasound energy received by the one or more sensors. Plots 321-326, derived in the same manner as plot 320, show the measure of intensity of the ultrasonic energy at later cardiac cycle times in sequence. Plots 310-316 correspond temporally with plots 320-326, respectively. However, it is noted that some systems may rapidly switch between A-mode and M-mode scans, such that the A-mode information and M-mode information represent different, but very close, points in time.

Measuring the intensity of the reflected ultrasound waves can provide information regarding the tissue density or other characteristic of the tissue. For example, denser tissue will typically reflect more ultrasound energy than similar but less dense tissue. Accordingly, an ultrasound sensor can measure more intense ultrasound energy reflected from denser sections of tissue and relatively less intense ultrasound energy reflected from less dense sections of tissue. The intensity of the ultrasound energy reflected from the first section 301 of cardiac tissue 309 is shown in the first section 306 of plot 320, while the second section 307 of plot 320 corresponds similarly with the second section 302 of plot 310 and the third section 308 of plot 320 corresponds similarly with the third section 303 of plot 310.

As shown in plot 310, the cardiac tissue is relatively consistent in dimension across the first, second, and third sections 301-303. Likewise, plot 320 shows that the intensity of the ultrasound energy reflected from the cardiac tissue across the first, second, and third sections 306-308 is consistent across the sections. Based on the consistently in received ultrasound energy across the first, second, and third sections 306-308 in plot 320, it can be concluded that the density of the corresponding sections of tissue is essentially the same across the first, second, and third sections 301-303.

Plot 311 shows ultrasound dimensional information from the same cardiac tissue 309 at a short time later (e.g., several milliseconds later) than the ultrasound echography information of plot 310. Plot 312 represents further ultrasound dimensional information of the cardiac tissue 309 a short time later than plot 311 and this chronological pattern is continued through plots 312-316 to represent various phases of at least one cardiac cycle. For example, plots 310 and 316 can correspond to a diastole phase while plot 313 can correspond to a systole phase. Plots 320-326 represent the different intensity levels of ultrasound energy measured from the cardiac tissue chronologically over the cardiac cycle.

Plot 311 shows that the cardiac tissue 309 has started to change in dimension relative to plot 310. The first section 301 and the third section 303 are shown in plot 311 to be thinner than in plot 310, and thinner than the adjacent second section 302 in the same plot 311. The second section 302 has maintained the larger thickness from plot 310. Plot 321 shows that the intensity of reflected ultrasound energy has increased for the first section 306 and the third section 308 relative to plot 320, and that the reflected ultrasound energy of the first section 306 and the third section 308 is greater than the reflected ultrasound energy of the second section 307. These changes in dimension and reflected energy are continued through plots 312 and 322.

In plot 313, the first section 301 and third section 303 are significantly reduced in thickness as compared to their thicknesses in plot 310, while the maximum thickness of second section 302 has remained substantially constant. Also, plot 323 shows that the first section 306 and the third section 308 have significantly higher levels of reflected ultrasound energy as compared to their original levels of reflected ultrasound energy of plot 320 and compared to the relatively constant level of reflected ultrasound energy from the second section 307 during the cardiac cycle. The changes in reflected ultrasound energy indicate that the cardiac tissue along the first section 301 and the third section 303 is becoming denser over plots 320-323, while the density of the second section 302 has not changed or changed in a substantially smaller degree as compared to level of ultrasound energy reflected from the first and third sections 306 and 308. The increase in density is indicative of the cardiac tissue 309 along the first and third sections compressing.

Plots 313-316 show that the first section 301 and third section 303 of cardiac tissue 309 are becoming thicker. Likewise, plots 324-326 show that the reflected ultrasound energy from the first and third sections 306 and 308 is decreasing, indicating that the tissue along these sections is becoming less dense, which is consistent with the cardiac tissue uncompressing. The consistent density of the second section 307 of cardiac tissue indicates that this tissue is not compressing and accordingly is not functional cardiac tissue. The M-mode view of the tissue in plots 310-316 shows that the thickness of the tissue is not changing, which is consistent with a lesion, fibrous tissue, scar tissue, or otherwise non-functioning tissue. The pattern of compressing and uncompressing of the first and third sections 306 and 308 of cardiac tissue is indicative of the tissue being functional cardiac tissue. The functional state of cardiac tissue can further be evaluated on the basis of whether the tissue compresses and decompresses in synchrony with a cardiac rhythm.

Cardiac rhythm information can be sensed during the sensing of ultrasound energy information. Cardiac rhythm information can be used to identify particular cardiac cycle phases. The diastole and systole phases are labeled in FIG. 3. In some embodiments, the cardiac cycle can be divided into a greater number of phases, such as atrial systole, isovolumetric ventricular compression, ventricular systole, and ventricular diastole phases. In some cases, each of the six plots 320-325 can be associated with different phases of a cardiac cycle, such that each plot represents the ultrasound information from different phases over a cardiac cycle. The sensed rhythm data can be an intrinsic electrical cardiac signal sensed directly or remotely from the heart. The sensed data can be an electrocardiogram (ECG), where the phase of atrial systole is typically regarded as beginning with the P wave and ventricular systole (sometimes referred to as cardiac systole or simply systole) begins at the start of the QRS complex. Diastole typically corresponds with the temporary flat lining of the ECG trace between the Q and T features. In various embodiments, the sensed cardiac rhythm information can be heart sounds, such as atrioventricular and semilunar valve closures indicating different phases of the cardiac cycle.

Collected ultrasound information can be associated with cardiac rhythm information to characterize the cardiac tissue in rhythm with the cardiac cycle. For example, the plots of FIG. 3 are arrayed sequentially over at least one cardiac cycle and are divided between different phases of the cardiac cycle. Furthermore, the systolic and diastolic phases are labeled. It would be expected that functional cardiac tissue would compress in the systolic phase and relax (uncompress) in the diastolic phase. In various embodiments, it can be determined whether the changes in received ultrasound energy (e.g., indicating changes in tissue density) are due to intrinsic contraction of the cardiac tissue or due to some other cause unrelated to the intrinsic cardiac contraction. For example, tissue can be determined to be compressing when an indicator of tissue density (e.g., the level of intensity of received ultrasound energy) increases in a systolic phase and decreases in a diastolic phase. Sections of tissue not fitting this profile can be determined to not be functioning. Sections of tissue fitting this profile can be determined to be functioning tissue, even if, for example, an electrical signal cannot be read directly from an electrode in contact with the tissue. As such, characterizing compressibility of cardiac tissue by identifying changes in the density of the tissue between phases of a cardiac cycle can indicate that the tissue is still functional despite electrophysiology techniques not being able to detect an electrical signature from the tissue which would otherwise indicate that the tissue was non-functional (e.g., fully lesioned by ablation).

Plots 320-326, as arrayed over at least one cardiac cycle, indicate that the second section 307 of cardiac tissue is not changing in the level of received ultrasound energy. The lack of change in ultrasound energy indicates that the density of the second section 307 of cardiac tissue is not changing between cardiac cycle phases and accordingly is not compressing over the at least one cardiac cycle. Specifically, the second section 307 has a consistent amplitude level over plots 320-326. The amplitude levels of the first and third sections 306, 308 demonstrate ultrasound intensity level changes, and thereby density changes, in the cardiac tissue 309 which is consistent with contraction. Furthermore, the density of the first and third sections 306 and 308 increasing during a systolic phase and decreases during a diastolic phase, as indicated by changing ultrasound intensity, matches a profile of functional cardiac tissue. The bulge of section 302 of plots 311-315 represents a lesion that does not change in density between systolic and diastolic phases of the at least one cardiac cycle. Tissue that does not change in density over at least one cardiac cycle matches a profile of non-functional tissue.

If the cardiac tissue 309 was previously lesioned, then the efficacy of the treatment can be assessed and further ablation therapy can be delivered as necessary. For example, the first section 301, second section 302, and third section 302 of cardiac tissue 309 may have been identified as being associated with improper electrical conduction leading to an arrhythmia. The three sections may have then been targeted for lesioning to block the improper conduction. The plots of FIG. 3 can represent the results of a first ablation treatment delivered to the cardiac tissue 309. Based on the lack of compressibility of the second section 302, it can be determined that the ablation treatment of this targeted tissue was successful. If the first and/or third sections 301 and 303 of cardiac tissue were previously treated with an ablation therapy, it can be determined that the ablation treatment did not inactivate these areas based on the compressibility of the first section 301 and the third section 303. These sections may still be functional and not fully lesioned even though the electrical function of the tissue may not be perceivable by an electrophysiology catheter sensing electrical activity.

If ablation therapy is being delivered to the second section 302 of cardiac tissue, and the state of the tissue is being monitored in real time as described herein, then the delivery can be stopped based on the lack of compression in the tissue over at least one cardiac cycle. Lesions can grow in size as more ablation therapy is delivered, and real-time monitoring of the state of the targeted tissue can determine when the size and shape of the lesion meets a target size and shape, and the therapy delivery can then be stopped or moved to another location. Additional ablation therapy can by delivered to the first section 301 and the third section 303 based on the compressibility of these sections to prevent the return of improper electrical activity associated with the tissue. Moreover, the further delivery of ablation therapy can be more closely targeted to the first section 301 and the third section 303. The cycle of assessing the compressibility of tissue and delivering ablation therapy to compressing tissue can be repeated until all targeted tissue is fully inactivated.

In some embodiments, if the compressibility of tissue is out of sync with the phases of the cardiac cycle (e.g., tissue compresses during the diastolic phase), then an ablation therapy can be delivered to eliminate the dyssynchrony or withheld to further investigate the cause of the abnormality.

Identification of a change in density indicative of compression can be automated in control circuitry to determine whether cardiac tissue is functioning and whether an ablation therapy inactivated tissue. For example, a change in intensity level of an ultrasound signal over different phases of one or more cardiac cycles can be identified by control circuitry. The change in intensity level can be compared to a predetermined threshold. The threshold can represent the change in amplitude (or other measure of intensity) expected for functional cardiac tissue between the different cardiac cycle phases. If the change is greater than or equal to the threshold, then tissue can be identified as compressing over a cardiac cycle. Tissue identified to be compressing can further be identified to be functional. If the change is less than the threshold, then the tissue can be identified to be compressing insufficiently to correspond to functional tissue (nevertheless, the tissue may be contracting to some degree and ablation therapy can be applied). If the intensity level of the ultrasound signal does not change between the phases of the one or more cardiac cycles, then it can be determined that the tissue is inactivated (and transmurally lesioned if previously treated with ablation). An output can be generated by control circuitry based on the determination of whether the cardiac tissue compresses. For example, if tissue associated with improper electrical activity is determined to compress, then an ablation therapy can be indicated for delivery (e.g., on a display) and/or the ablation therapy can be automatically delivered by an ablation system. The output generated on the display may positively or negatively indicate whether the tissue compressed and/or whether ablation therapy was successful. In some cases, the compressibility of tissue can be monitored concurrent with, or in between, delivery of ablation therapy. The ablation therapy can then be stopped when it is determined that the tissue no longer compresses over at least one cardiac cycle.

It is noted that the intensity of reflected ultrasound energy can change based on the distance between the ultrasound sensor and the tissue reflecting the ultrasound waves. Cardiac tissue is usually moving due to the constant dynamic function of the heart. Even inactivated cardiac tissue moves during a cardiac cycle and ultrasound energy measured from the tissue in A-mode will change over a cardiac cycle. These changes could present themselves as changes in tissue density, even if the density of the tissue does not actually change during the cardiac cycle. However, control circuitry can correct for the movement of tissue by various techniques. By monitoring tissue in an M-mode, dimensional and movement information can be collected. A signal indicative of the intensity of reflected ultrasound energy can be normalized in synchrony with the wall motion identified from an M-mode scan or the changes in the intensity of an ultrasound signal (e.g., the signal amplitude in A-mode) can otherwise be corrected or canceled out based on the wall motion known from the M-mode scan. In some embodiments, the distance between the ultrasound sensor and the tissue can be tracked by scanning in M-mode, and changes in the distance can be used to correct or cancel out changes in the signal intensity due to the distance changes. As such, various embodiments can include processing the signal containing the ultrasound intensity information to reduce or eliminate changes in the signal due to motion of the tissue relative to the sensor. Such processing can highlight changes in the signal due to changes in tissue density.

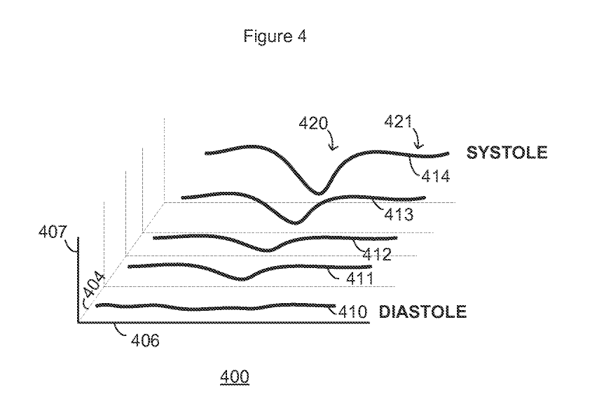

FIG. 4 illustrates a composite plot 400 of ultrasound intensity information associated with a plurality of different cardiac cycle phases. The abscissa axis 406 of the plot 400 represents a linear dimension across which the ultrasound echography system scans (e.g., a left-to-right scan over a tissue section). The ordinate axis 407 represents the intensity of the ultrasound energy received by the one or more sensors (e.g., amplitude in an A-mode scan). The information of the composite plot 400 can be collected in the same manner as plots 320-326 of FIG. 3. The ultrasound intensity traces 410-414 of FIG. 4 are arrayed over the time axis 404 to show the progression of the intensity changes over different cardiac cycle phases. Cardiac rhythm information can also be collected and processed to identify particular phases within the cardiac cycle. Several phases in FIG. 4 are labeled as diastole and systole to show that, at the same time the traces were sensed, the heart was in a systole or diastole phase. Based on the change in the level of intensity of the ultrasound signal between the cardiac cycle phases, tissue that is associated with a lack of change in density can be identified as inactive (e.g., lesioned, scar tissue, or other state as referenced herein). In some embodiments, a lesion 420 or other tissue state can be graphically identified based on a consistent or changing ultrasound intensity level (e.g., in the plot 400 or on cardiac map). Non-lesioned tissue 421 can also be identified from the progression of ultrasound intensity changes between cardiac cycle phases. As such, the identification of lesioned and non-lesioned areas (or active and inactive areas, or compressing and non-compressing tissues) can be identified by overlaying ultrasound intensity information from different cardiac cycle phases. FIG. 4 also demonstrates that changes in tissue density can be identified by comparing ultrasound intensity levels from different phases of a cardiac cycle to one another. Such comparison can be done graphically, as in FIG. 4, or numerically.

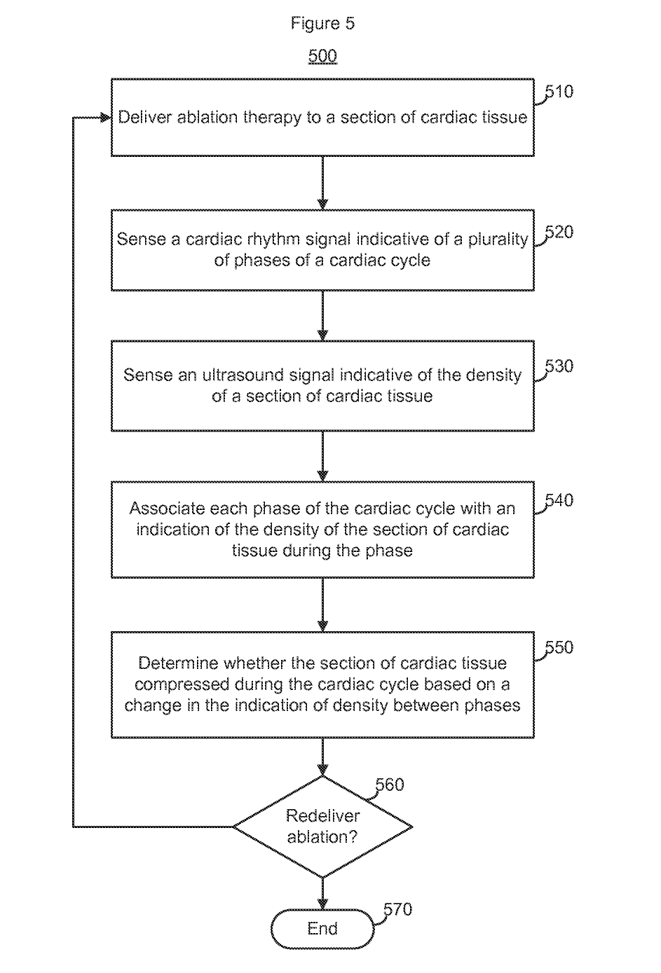

FIG. 5 illustrates a flow chart 500 of a method for managing tissue ablation based on tissue compressibility. The method includes delivering 510 an ablation therapy to a section of cardiac tissue. The method further includes sensing 520 a cardiac rhythm signal indicative of a plurality of phases of a cardiac cycle. Sensing 520 a cardiac rhythm signal can include sensing one or more electrical cardiac signals (e.g., an ECG) or sensing one or more sounds (e.g., heart valve sounds), among other options. Multiple phases of a cardiac cycle can be identified from the sensed 520 signal, as discussed herein. Such cardiac cycle phases can comprise systole and diastole phases, although other options for cardiac phases are possible.

The method further includes sensing 530 an ultrasound signal. The ultrasound signal can be indicative of the density of a section of cardiac tissue. In some cases the intensity level of the signal can be correlated to the density of the tissue, such that a higher intensity level of the signal can correspond to denser tissue while a lower intensity level of the signal can correspond to softer tissue. The ultrasound signal may be sensed 530 over one or more cardiac cycles. The ultrasound signal may be sensed 530 from a particular section of cardiac tissue, such as a small wall section of an atrium or ventricle. The cardiac signal may be processed in accordance with an A-mode scan which allows changes in the amplitude of the signal to be identified. Sensing 530 may further include performing one or more scans in accordance with M-mode operation to identify dimensional aspects of the tissue which may be used, for example, in correcting for or canceling wall motion effects. The ultrasound signal may be sensed by one or more ultrasound transducers located on the distal end of a catheter configured to be introduced into the heart. In some embodiments, the ultrasound transducer may be on the same catheter on which the sensing element that senses 520 the cardiac rhythm signal is disposed.

The ultrasound signal may be sensed 530 over the same one or more cardiac cycles for which the cardiac rhythm signal is sensed 520, however the timing of the cardiac phases may already be established based on a previously sensed 520 cardiac rhythm signal. The ultrasound information may be sensed 530 continuously over the one or more cardiac cycles (e.g., constant scanning by one or more ultrasonic transducers), or sensed 530 in discrete sets to represent snapshots over the one or more cardiac cycles. For example, the ultrasound signal may only be sensed 530 or otherwise collected in discrete samples corresponding to specific cardiac phases identified based on the sensed 520 cardiac rhythm signal (e.g., sampling ultrasound echo energy only during the end of the diastolic phase and the peak or end of the systolic phase). It is noted that sensing 520 the cardiac rhythm signal and sensing 530 the ultrasound signal steps may be performed concurrently for the same one or more cardiac cycles. As such, although the method can be performed in the chronological order of the flowchart 500, the steps can be arranged to be performed concurrently and/or in various other orders.

The method further includes associating 540 each phase of the cardiac cycle with an indicator of the density of the section of cardiac tissue as indicated by the ultrasound signal during the phase. Associating 540 can include arranging the ultrasound signal information in discrete phases along the cardiac cycle for the purpose of displaying the information (e.g., as in FIG. 3). The indicator of density can be a portion of the ultrasound signal, a measure of the intensity of the ultrasound signal such as amplitude, a numerical value, and/or some other information derived from the ultrasound signal and indicative of the density of the tissue from which the ultrasound waves reflected. Associating 540 can include determining in which phase of a cardiac cycle a particular portion of the ultrasound signal was sensed 530. For example, associating 540 a phase of a cardiac cycle with the density level can include determining that a particular portion of the ultrasound signal, indicative of density, was sensed during systole and that a different portion of the ultrasound signal, sensed at a different time, was sensed during diastole. In some cases, the ultrasound signal is selectively sensed 530 or portions of which are retained in memory based on correspondence to different cardiac phases. For example, different portions of an ultrasound signal can be associated with different cardiac phases based on the signal only being sensed or otherwise sampled for those different phases. Associating can include saving an intensity level or other ultrasound information in memory along with corresponding cardiac rhythm information (e.g., by saving pointers or other indicators along with particular ultrasound information noting the phase in which the particular ultrasound information was sensed).

The method further includes determining 550 whether the section of cardiac tissue compressed during the cardiac cycle based on a change in the indication of density between the phases. As discussed herein, a change in density of the tissue over a cardiac cycle, as indicated by a corresponding change in intensity of reflected ultrasound energy, can indicate compressing tissue while a lack of a change in density over the cardiac cycle can indicate non-compressing tissue. Cardiac tissue that does not compress over a cardiac cycle can be indicative of a transmural lesion. In some cases, a threshold is used to distinguish between compressing and non-compressing tissue. For example, a difference in a measure of ultrasound intensity (e.g., amplitude of an A-mode scan) between two phases of a cardiac cycle (e.g., a first phase associated with systole and a second phase associated with diastole) greater than a predetermined threshold can indicate that the tissue compressed during the cardiac cycle. A difference in ultrasound intensity less than the threshold can indicate a lack of normal compression otherwise associated with functional cardiac tissue. A lack of change in ultrasound intensity can indicate no compression of the tissue which can be indicative of a transmural lesion.

In some embodiments, determining 550 whether the cardiac tissue compressed includes determining whether the indication of density (e.g., ultrasound intensity) of the tissue changed in synchrony with the cardiac cycle. For example, if the cardiac cycle is divided into a diastolic phase and a systolic phase, then it can be determined whether the section of cardiac tissue showed relatively greater density during the systolic phase (e.g., as indicated by greater ultrasound echo energy) and showed relatively less density during the diastolic phase (e.g., as indicated by lesser ultrasound echo energy). If the ultrasound information of the sampled section of cardiac tissue as associated with the cardiac rhythm information fits this pattern, then the determination 550 can conclude that the sampled section of tissue is compressible and therefore functional (and not fully lesioned). If the ultrasound information of the sampled section of cardiac tissue as associated with the cardiac rhythm information does not fit this pattern, then the determination 550 can conclude that the sampled section of tissue is not compressible and therefore non-functional (e.g., by way of a lesion). For example, the tissue may be determined 550 to be non-compressing if the ultrasound intensity level of the tissue section is substantially consistent over all of the cardiac phases or if the changes in ultrasound intensity level is inconsistent with patterns indicative of functional cardiac tissue (e.g., tissue may be identified as not compressible if there is greater ultrasound energy received during a diastolic phase and less ultrasound energy received during a systolic phase).

If it is determined 550 that the tissue is not compressing, then the method can end 570. In some cases, a determination can be made over whether a particular section of cardiac tissue was successfully ablated based on the compressibility of the tissue. For example, if the particular section of tissue is determined 550 to not compress then it can be concluded that the ablation therapy previously or currently being delivered has fully lesioned the tissue. In this case, an ablation therapy can be stopped if it is being delivered and/or an indication of the completion of the lesion can be generated (e.g., a mark on a display and/or an audible noise). If a particular section of tissue is determined 550 to compress, then it can be concluded that an ablation therapy previously or currently being delivered failed to fully lesion the tissue. In this event, additional ablation therapy can be delivered 510 until compression is no longer detected for at least one cardiac cycle.

The determination 550 of compressibility of tissue can be used to control delivery of an ablation therapy. If ablation therapy is currently being delivered or is scheduled for redelivery, then the ablation therapy can be stopped or the redelivery canceled if it is determined that the tissue no longer compresses. In some cases, the steps of the method of FIG. 5 can be performed repeatedly or continuously until it is determined that the section of tissue was fully ablated (e.g., with a transmural ablation) based on the section of cardiac tissue no longer compressing along with the cardiac cycle.

In some embodiments, the state of tissue can be determined and an output generated by control circuitry based on the state. For example, non-functional tissue that is fully ablated (e.g., transmurally) can be identified based on a lack of compression over a cardiac cycle and a lack of electrical signature sensed from the tissue. Stunned, swollen (e.g., edema), or otherwise temporarily affected tissue can be identified based on the tissue compressing between different phases of a cardiac cycle and a lack of electrical signature sensed from the tissue. Fully functional tissue can be identified based on the tissue compressing between different phases of a cardiac cycle and the presence of an electrical signature sensed from the tissue.

It is noted that various modifications can be made to the steps and/or the flowchart 500 of FIG. 5. In various embodiments, various steps of the method can be performed simultaneously or sequentially, such as sensing 520 the cardiac rhythm signal and sensing 530 the ultrasound signal. In some cases, each of the steps of the method can be performed continuously or intermittently, for example, until it is determined that the targeted tissue is fully ablated. In some embodiments, sensing 520 the cardiac rhythm signal, sensing 530 the ultrasound signal, associating 540, and determining 550 compressibility can be performed without ablation to profile the section of tissue. For example, these steps, and/or any other steps referenced herein can be performed to assess the function of tissue without a preceding and/or subsequent ablation therapy being delivered. Such assessment may be to determine the state of cardiac tissue following infarction, arrhythmia (e.g., atrial fibrillation), or other events. The tissue being evaluated for compressibility could be scar tissue created by a previous injury, fibrous tissue, tissue associated with myocardial infarction, or tissue subject to any event or condition that could potentially compromise the contractility function of the tissue, where the changes in density of the tissue over one or more cardiac cycles can indicate the compressibility of the tissue. As such, various embodiments of the present disclosure can concern characterizing cardiac tissue based on whether the density of the tissue changes over one or more cardiac cycles. Diseased, scarred, fibrous, or otherwise non-functioning tissue will generally have a consistent level of density over a cardiac cycle because such tissue does not compress, while healthy cardiac tissue will change in density over the cardiac cycle, and whether the tissue changes in density can be used to discriminate different tissue states as described herein.

Various applications of the present disclosure can guide therapy intensity based on the detected compressibility of tissue. For example, an area of tissue can contain overlapping layers of conductive tissue and non-conductive fibrous tissue. If the area is targeted for ablation (e.g., as part of a conduction block procedure), the state of the tissue can be assessed as described herein to determine whether the density of the tissue changes with the cardiac cycle. If the changes in density indicate contracting tissue, then ablation therapy can be delivered. If no changes in density are detected, then ablation therapy can be withheld. If the characterization of the tissue indicates that one or more portions (e.g., one or more layers) do not contract (e.g., the tissue is determined to be fibrous) and one or more other portions (e.g., one or more other layers of the same area of tissue) do contract or otherwise are indicated to change in density, then ablation therapy can be delivered because the contracting tissue can still conduct electrical energy and propagate an arrhythmia. However, the intensity of the ablation therapy delivery may be increased for this area because the one or more fibrous tissue portions may insulate the one or more conductive tissue portions from the ablation therapy. In some cases, the identification of overlapping layers of fibrous and non-conductive tissue (or overlapping layers of contracting and non-contracting tissue) may be automatically performed by control circuitry. In some of these cases, an indication of the presence of the mixed layers can be displayed on a screen by control circuitry. In some embodiments, the displayed indication may include a recommendation to increase the intensity of therapy delivery. In some embodiments, control circuitry may automatically increase the intensity of ablation therapy based on the detection of overlapping layers of fibrous and non-conductive tissue (or overlapping layers of contracting and non-contracting tissue) for an area of tissue.

It is noted that the steps of the method of FIG. 5, and/or any steps referenced herein, can be performed by control circuitry. For example, the steps of the method of FIG. 5 and/or any other steps referenced herein could be implemented by the system 100 of FIG. 1 in an automated manner by the control circuitry of FIG. 2. Likewise, any of the plots of FIGS. 3 and 4 and/or similar plots could be generated and displayed using the system 100 and control circuitry of FIG. 2 or any modifications thereof to characterize tissue and guide therapy.

The techniques described in this disclosure, including those of FIGS. 1-5 and those attributed to a system, control circuitry, processor, or various constituent components, may be implemented wholly or at least in part, in hardware, software, firmware or any combination thereof. A processor, as used herein, refers to any number and/or combination of a microprocessor, a digital signal processor (DSP), an application specific integrated circuit (ASIC), a field-programmable gate array (FPGA), microcontroller, discrete logic circuitry, processing chip, gate arrays, and/or any other equivalent integrated or discrete logic circuitry. “Control circuitry” as used herein refers to at least one of the foregoing logic circuitry as a processor, alone or in combination with other circuitry, such as memory or other physical medium for storing instructions, as needed to carry about specified functions (e.g., a processor and memory having stored program instructions executable by the processor for determining whether a section of cardiac tissue compressed during a cardiac cycle based on a change in an indication of density of the section over different phases of the cardiac cycle based on an ultrasound signal). The functions referenced herein may be embodied as firmware, hardware, software or any combination thereof as part of control circuitry specifically configured (e.g., with programming) to carry out those functions, such as in means for performing the functions referenced herein. The steps described herein may be performed by a single processing component or multiple processing components, the latter of which may be distributed amongst different coordinating devices. In this way, control circuitry may be distributed between multiple devices. In addition, any of the described units, modules, subsystems, or components may be implemented together or separately as discrete but interoperable logic devices of control circuitry. Depiction of different features as modules, subsystems, or units is intended to highlight different functional aspects and does not necessarily imply that such modules or units must be realized as hardware or software components and/or by a single device. Rather, specified functionality associated with one or more module, one or more subsystems, or one or more units, as part of control circuitry, may be performed by separate hardware or software components, or integrated within common or separate hardware or software components of control circuitry.

When implemented in software, the functionality ascribed to the systems, devices, and control circuitry described in this disclosure may be embodied as instructions on a physically embodied computer-readable medium such as RAM, ROM, NVRAM, EEPROM, FLASH memory, magnetic data storage media, optical data storage media, or the like, the medium being physically embodied in that it is not a carrier wave, as part of control circuitry. The instructions may be executed to support one or more aspects of the functionality described in this disclosure.

Although the embodiments referenced herein are described in the context of assessing the compressibility of cardiac tissue, the systems and methods referenced herein can be applied to profiling other areas of the body. For example, the systems and methods of this disclosure could be used for profiling or treating the prostate, brain, gall bladder, uterus, esophagus, and/or other regions in the body. Non compressing tissue can be identified as lesioned or otherwise non-functional tissue while compressing tissue can be identified as functioning tissue.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as falling within the scope of the claims, together with all equivalents thereof.

What is claimed is:

1. A system comprising:
at least one catheter having a distal end configured to be introduced into the heart;
at least one ultrasound sensor on the distal end of the at least one catheter, the at least one ultrasound sensor configured to output a first signal indicative of the intensity of ultrasound energy received by the ultrasound sensor from a section of cardiac tissue, the intensity of the ultrasound energy indicative of the density of the section of cardiac tissue;
a sensor configured to output a second signal indicative of a plurality of different phases of at least one cardiac cycle; and
control circuitry configured to associate each phase of the plurality of different phases of the at least one cardiac cycle with the intensity level of ultrasound energy received by the ultrasound sensor from the section of cardiac tissue during the phase, determine whether the section of cardiac tissue compressed during the at least one cardiac cycle based on a difference between the intensity levels of ultrasound energy associated with different phases of the at least one cardiac cycle, and generate an output based on the determination of whether the section of cardiac tissue compressed.

2. The system of claim 1, wherein the control circuitry is configured to determine that the section of cardiac tissue compressed if the intensity level of ultrasound energy associated with a systolic phase is greater relative to the intensity level of ultrasound energy associated with a diastolic phase and that the section of cardiac tissue did not compress if the intensity level of ultrasound energy associated with the systolic phase is similar to the intensity level of ultrasound energy associated with the diastolic phase.

3. The system of claim 1, wherein the difference between the intensity levels of ultrasound energy associated with different phases of the at least one cardiac cycle is indicative of a change in density of the section of cardiac tissue between the different phases of the at least one cardiac cycle.

4. The system of claim 1, further comprising a display, wherein the control circuitry is configured to generate an indication on the display of the state of the section of cardiac tissue based on the determination of whether the section of cardiac tissue compressed.

5. The system of claim 1, further comprising a display, wherein the control circuitry is configured to generate a cardiac map on the display and highlight the section of cardiac tissue on the cardiac map based on the control circuitry determining that the section did not compress.

6. The system of claim 1, further comprising an ablation element configured to output a cardiac ablation therapy, wherein the control circuitry is configured to determine whether the section of cardiac tissue was ablated by the cardiac ablation therapy based on the determination of whether the section of cardiac tissue compressed.

7. The system of claim 1, further comprising an ablation element configured to output a cardiac ablation therapy, wherein the control circuitry is configured to repeatedly or continuously deliver the cardiac ablation therapy with the ablation element to the section of cardiac tissue until the control circuitry determines that the section of cardiac tissue no longer compresses.

8. The system of claim 1, wherein:
the first signal is indicative of the level of ultrasound energy received by the ultrasound sensor from an additional section of cardiac tissue that is adjacent to the section of cardiac tissue; and
the control circuitry is configured to associate each phase of the plurality of different phases of the at least one cardiac cycle with the intensity level of ultrasound energy received by the at least one ultrasound sensor from the additional section of cardiac tissue during the phase and determine whether the section of cardiac tissue compressed during the at least one cardiac cycle relative to the additional section of cardiac tissue based on the intensity levels of ultrasound energy associated with the different phases of each of the section and the additional section of cardiac tissue.

9. The system of claim 1, wherein the control circuitry is configured to process the first signal in accordance with A-mode ultrasound operation.

10. The system of claim 1, wherein the control circuitry is configured to selectively sample the first signal only during respective portions of the plurality of different phases based on the second signal to associate each phase of the plurality of different phases with the intensity level of ultrasound energy received by the at least one ultrasound sensor during the respective portion of the phase.

11. The system of claim 1, wherein the control circuitry is configured to reduce or eliminate changes in the intensity level of the ultrasound energy of the first signal due to wall motion of the section of cardiac tissue.

12. A method of assessing cardiac ablation, the method comprising:
delivering an ablation therapy to a section of cardiac tissue, the ablation therapy delivered by a catheter to the section of cardiac tissue;
sensing a first signal indicative of a plurality of different phases of at least one cardiac cycle with a sensor;
sensing a second signal with one or more ultrasound sensors within the heart over the plurality of different phases of the at least one cardiac cycle, the second signal indicative of the density of a section of cardiac tissue;
associating each phase of the plurality of different phases of the at least one cardiac cycle with an indication of the density of the section of cardiac tissue during the phase based on the second signal;
determining whether the section of cardiac tissue compressed during the at least one cardiac cycle based on a change in the indication of density of the section of cardiac tissue over the plurality of different phases of the at least one cardiac cycle; and
determining whether the section of cardiac tissue was ablated by the delivery of the ablation therapy based on whether the section of cardiac tissue compressed during the at least one cardiac cycle.

13. The method of claim 12, wherein determining whether the section of cardiac tissue compressed comprises:
determining that the section compressed if the density of the section associated with the systolic phase is indicated to be greater than the density of the section associated with the diastolic phase; and
determining that the section did not compress if the density of the section associated with the systolic phase is indicated to be similar to the density of the section associated with the diastolic phase.

14. The method of claim 12, further comprising redelivering the ablation therapy to the section of cardiac tissue if it is determined that the section of cardiac tissue was not ablated by the delivery of the ablation therapy.

15. The method of claim 12, wherein the plurality of different phases of the at least one cardiac cycle comprises at least a diastole phase and a systole phase.

16. The method of claim 12, wherein sensing the second signal comprises selectively sensing the second signal only during respective portions of the plurality of different phases based on the first signal to associate each phase of the plurality of different phases with the indication of the density of the section of tissue during the phase.

17. The method of claim 12, wherein the density of the section of cardiac tissue is indicated by the intensity level of ultrasound energy received by the second sensor as reflected from the section of cardiac tissue.

18. A system comprising:
at least one catheter having a distal end configured to be introduced into the heart;
at least one ultrasound sensor on the distal end of the at least one catheter, the at least one ultrasound sensor configured to output a first signal indicative of the intensity level of ultrasound energy received by the at least one ultrasound sensor from a section of cardiac tissue, the intensity level of ultrasound energy received by the at least one ultrasound sensor indicative of the density of the section of cardiac tissue;
a sensor configured to output a second signal indicative of a plurality of different phases of at least one cardiac cycle;
a display; and
control circuitry configured to associate each phase of the plurality of different phases of the at least one cardiac cycle with the intensity level of ultrasound energy received by the at least one ultrasound sensor from the section of cardiac tissue within the phase and generate an output on the display representing the intensity levels of ultrasound energy as associated with the different phases of the plurality of different phases of the at least one cardiac cycle, the output on the display indicative of whether the section of cardiac tissue compressed during the at least one cardiac cycle.

19. The system of claim 18, wherein the output on the display comprises overlaid signal traces, each signal trace of the overlaid signal traces representing the intensity level of ultrasound energy as associated with a respective phase of the plurality of different phases of the at least one cardiac cycle.

20. The system of claim 18, wherein the control circuitry is further configured to label at least one phase of the plurality of different phases as a systolic phase and label at least one other phase of the plurality of different phases as a diastolic phase in the output generated on the display based on the second signal.
